(19) United States
(12) Patent Application Publication
(10) Pub. No.: US 2021/0071287 A1
(43) Pub. Date: Mar. 11, 2021

(54) DUPLEX STAINLESS STEELS AND USES THEREOF

(71) Applicant: SAIPEM S.p.A., San Donato Milanese (IT)

(72) Inventors: Giulio Paci, San Donato Milanese (IT); Mara Quattrocchi, San Donato Milanese (IT); Lino Carlessi, San Donato Milanese (IT); Alberto Serrafero, San Donato Milanese (IT)

(21) Appl. No.: 16/771,951

(22) PCT Filed: Dec. 20, 2018
(86) PCT No.: PCT/IB2018/060408

§ 371 Date: Jun. 11, 2020

(30) Foreign Application Priority Data
Dec. 22, 2017 (EP) 17210463.0

(51) Int. Cl. C22C 38/52; C22C 38/42; C22C 38/44; C22C 38/02; C22C 38/00; B01J 19/02; C07C 273/02

(52) U.S. Cl. CPC C22C 38/52; C22C 38/42; C22C 38/44; C22C 38/02; C07C 275/02; C22C 38/002; B01J 19/02; C07C 273/02; C22C 38/001

(57) ABSTRACT

A duplex stainless steel for use in a urea production plant and/or in a urea production process, containing in weight percentage (% w): C 0.03 or less; Si 0.5 or less; Mn 2.5 or less; Cr from more than 30.0 to 35.0; Ni from 5.5 to 8.0; Co from 0.01 to 0.8; Mo from 2.0 to 2.5; W 2.5 or less; N from 0.3 to 0.6; Cu 1.0 or less; and having one or more of: Ca 0.0040 or less; Mg 0.0040 or less; one or more rare-earth elements in a total amount of 0.1 or less; the balance being Fe and impurities; and satisfying the relationship: Z=1.062 (Ni+Co)+4.185 Mo is between 14.95 and 19.80.

### PRIORITY CLAIM

This application is a national stage application of PCT/IB2018/060408, filed on Dec. 20, 2018, which claims the benefit of and priority to European Patent Application No. 17210463.0, filed on Dec. 22, 2017, the entire contents of which are each incorporated by reference herein.

### TECHNICAL FIELD

The present disclosure relates to the use of a duplex stainless steel in relatively highly corrosive urea environments containing ammonia carbamate at relatively high temperatures and pressures.

The disclosure thus also relates to the use of a duplex stainless steel in a urea plant (i.e., plant for production of urea), and specifically in an apparatus, equipment or device (or a part thereof) which is exposed to concentrated ammonium carbamate at relatively high temperature.

The disclosure also relates to an apparatus, equipment or device of a urea production plant or used in a urea production process, comprising at least a part made of a corrosion resistant duplex stainless steel.

The disclosure also relates to a plant and a process for the production of urea comprising at least one apparatus, equipment or device having at least a part made of a duplex stainless steel, and to a method of revamping an existing urea production plant by replacing at least a part of an apparatus, equipment or device of the plant with a part made of a duplex stainless steel.

### BACKGROUND

Duplex stainless steels are a family of stainless steels characterized by a two-phase microstructure consisting of grains of austenite and ferrite in roughly equal proportions.

The austenite-ferrite structure gives this family of stainless steels a combination of favorable properties, in particular good mechanical strength and excellent resistance to corrosion.

However, commonly available grades of duplex stainless steels, even if generally exhibiting a good corrosion resistance, are not suitable for use under very severe conditions, such as in a urea production plant and specifically in a high pressure section of a urea plant.

As it is known, urea production is based on a relatively high-temperature, relatively high-pressure reaction of carbon dioxide and ammonia to form ammonium carbamate, and a subsequent dehydration reaction of the ammonium carbamate to form urea and water.

In a typical urea production plant (urea plant), these processes are generally carried out in a urea synthesis reactor operating at relatively high pressure and relatively high temperature; the aqueous urea solution produced in the synthesis reactor is then progressively concentrated, with recover of unconverted reagents, in one or more recovery sections, for example in a high-pressure section, a medium-pressure section and a low-pressure section; finally, the urea is solidified in a finishing section, which normally includes a granulator or a prilling tower.

Industrial level processes and plants for the production of urea are today largely based on stripping processes: the synthesis solution exiting from the reactor is subjected to heating at relatively high pressure (substantially the same pressure of the reactor) and the ammonium carbamate decomposes into ammonia and carbon dioxide in the liquid phase; part of the ammonia, together with carbon dioxide, passes from the liquid phase to the gas phase. The gas phase collected from the stripper is condensed and recycled to the reactor.

In some industrial processes, ammonia is used as a stripping agent (ammonia-stripping process), or the stripping is performed only by supplying heat, without any stripping agent (self-stripping process, or thermal stripping process).

In other industrial processes, such as the so-called CO2-stripping process, the stripping agent is gaseous carbon dioxide.

In a urea synthesis plant operating according to the ammonia-stripping process or the self-stripping process, corrosion resistance is an essential feature.

In particular, the ammonia-stripping process and the self-stripping process have a high pressure section, basically comprising the urea synthesis reactor and the urea stripper (as well as auxiliary equipment and devices), where the corrosion resistance is most important, due to the presence of the intermediate compound ammonium carbamate solution.

The ammonia-stripping process and the self-stripping process are preferably performed at a maximum temperature of 185° C. or higher (more preferably at 190° C. or higher, in particular at 205° C. or higher and preferably in the range 205° C.-215° C.); at a maximum pressure of 150 bar or higher (preferably of 156 bar or higher and more preferably of about 160 bar or higher); and with a NH3/CO2 molar ratio (so-called N/C ratio) in the range 3.2-3.6.

For example, stripping processes of the type described just above, operating at such conditions, are used in the so-called “Snamprogetti Urea Technology”, which is known to the skilled person being widely used worldwide and often cited in technical texts and papers.

Thus, at least some apparatus, equipment or device of the urea plant, in particular of the high pressure section thereof, such as (but not only) the urea stripper, operate under processing conditions which are relatively highly corrosive, particularly due to the presence of a hot and concentrated carbamate solution at relatively high temperatures (185° C.-205° C. and over) and pressures (150 bar or higher).

Similar problems are however also present in other kinds of urea production plants also having a high pressure section.

Therefore, the high pressure section of a urea plant (in particular, but not only, in a urea plant operating according to the ammonia-stripping process or the self-stripping process) usually requires addition of a certain amount of oxygen (typically in form of a stream of inerts also including oxygen) for passivating the metal surfaces (especially, but not only, if made of austenitic stainless steels). Use of oxygen in the high pressure section can however increase the risk to originate potentially explosive mixture and therefore there is a concern in terms of safety.

In order to reduce the use of passivation gas streams and/or to improve corrosion resistance, duplex stainless steels have been proposed for use in urea production plants.

For example, PCT Patent Application No. WO95/00674 discloses the use of a particular duplex stainless steel, the so called super duplex stainless steel sold under the trademark Safurex®, for making some equipment of urea plants.

However, the super duplex stainless steels of PCT Patent Application No. WO95/00674, when used in a carbamate environment, may be not fully effective at relatively very high temperatures (higher than 180° C.-200° C.), such as common operation temperatures of ammonia-stripping or self-stripping processes. Use of known duplex stainless steels is thus confined to CO2-stripping processes.

PCT Patent Application No. WO2014/180761 discloses a shell-and-tube urea stripper, to be specifically used in an ammonia-stripping or self- stripping process, having a bundle of tubes made of certain duplex stainless steels, namely of the Safurex® steel 29Cr-6.5Ni-2Mo—N (ASME Code 2295-3 and UM S32906), or the DP28W™ steel 27Cr-7.6Ni-1Mo-2.3W-N (ASME Code 2496-1 and UM S32808).

Also PCT Patent Application No. WO2017013180A1, PCT Patent Application No. WO2017013181A1 and PCT Patent Application No. WO2017014632A1 disclose duplex stainless steels generally suggested for use in urea plants under relatively high temperature and relatively high pressure conditions.

It can be appreciated that all the prior art documents cited above disclose duplex stainless steels which do not contain cobalt.

PCT Patent Application No. WO2006/049572 discloses a duplex stainless steel alloy which contains also cobalt and shows relatively high strength, good corrosion resistance, good workability and which is weldable. The proposed alloys are intended for use in the onshore and offshore sectors of the oil and gas industry, while uses under more severe corrosive conditions (such as in a urea plant/process) are not mentioned.

Therefore, even if duplex stainless steels are known which have relatively good corrosion resistance and are allegedly suitable for use also in a urea production plant, there is still a need for other, possibly more corrosion resistant duplex stainless steels which are suitable for use in any urea environments (i.e., in any kind of urea production plants/processes) and specifically in an apparatus operated at relatively high temperatures in contact with relatively very corrosive fluids (containing ammonium carbamate) and also under oxygen-free conditions, such as for instance (but not only) the high pressure strippers (operated at pressure of 150 bar and more) used in an ammonia-stripping process or a self-stripping process.

### SUMMARY

Accordingly, it is an object of the present disclosure to provide a duplex stainless steel suitable for overcoming certain of the problem foregoing described of certain of the prior art.

In particular, it is an object of the present disclosure to provide duplex stainless steels which are specifically and fully suitable to be used in a urea environment (i.e., in contact with a fluid comprising ammonium carbamate, such as a concentrated ammonium carbamate solution), and also at temperatures of at least 185° C., preferably of at least 190° C. and more preferably of 205° C. and more, even under oxygen-free conditions.

It is also a specific object of the disclosure to provide corrosion resistant duplex stainless steels which are suitable for use in any urea environments (i.e., in any kind of urea production plants/processes), and specifically in an apparatus (such as a high pressure stripper) used in an ammonia-stripping process or a self-stripping process, and thus operating at a maximum temperature of 185° C. or higher (preferably at 190° C. or higher, in particular at 205° C. or higher and preferably in the range 205° C.-215° C.); and/or at a maximum pressure of 150 bar or higher (preferably of 156 bar or higher and more preferably of about 160 bar or higher); and/or with a NH3/CO2 molar ratio (so-called N/C ratio) in the range 3.2-3.6.

The present disclosure accordingly relates to a duplex stainless steel for use in a urea production plant and/or in a urea production process as disclosed herein. The disclosure also relates to an apparatus, equipment or device, in particular of a urea production plant or used in a urea production process, comprising at least a part made of a corrosion resistant duplex stainless steel as disclosed herein. The disclosure also relates to a plant and a process for the production of urea comprising at least one such apparatus, equipment or device having at least a part made of a duplex stainless steel as disclosed herein. The disclosure further relates to a method of revamping an existing urea production plant by replacing at least a part of an apparatus, equipment or device of the plant with a part made of a duplex stainless steel as disclosed herein.

The duplex stainless steels of the disclosure are specifically characterized by a combination of nickel (Ni), cobalt (Co) and molybdenum (Mo): wherein it has been recognized that such three elements, used together according to specific composition rules, have an unexpected combined effect on the corrosion resistance as well as on other favorable material properties.

It should be appreciated that it has been found that these three elements (Ni, Co, Mo) effectively increase corrosion resistance of a duplex stainless steel (having the particular composition of the disclosure) if each element is used in a specific content range and the contents of the three elements are linked to one another by a composition parameter Z which ranges between a minimum value Zₘᵢₙ and a maximum value Zₘₐₓ.

In particular, the duplex stainless steels of the disclosure have a composition parameter Z ranging between 14.95 and 19.80, preferably between 14.95 and 19.00, more preferably between 14.95 and 18.00, even more preferably between 14.95 and 17.50.

Composition parameter Z is a parameter representative of the combined contents of Ni, Co, Mo and defined by formula (I):

Z=1.062 (Ni+Co)+4.185 Mo (I)
where Ni, Co, Mo indicate the weight percentage of Ni, Co, Mo respectively.

According to the disclosure:

14.95≤Z≤19.80

In other words, the duplex stainless steels of the present disclosure have the particular compositions of the disclosure also exhibit an excellent corrosion resistance (in particular, in urea environments) if parameter Z is maintained in the ranges defined above if components Ni, Co and Mo are used in amounts which satisfy formula (II):

Zₘᵢₙ≤[1.062(Ni+Co)+4.185 Mo]≤Zₘₐₓ (II)
where:
   Ni, Co, Mo indicate the weight percentage of Ni, Co, Mo respectively
   Zₘᵢₙ=14.95
   Zₘₐₓ=19.80

Experimental tests confirm that duplex stainless steels according to the disclosure, (i.e., having a combined content of Ni, Co and Mo as previously defined), satisfying formula (II), have a corrosion rate in urea environments (containing ammonium carbamate) significantly lower than prior art materials, even at relatively high temperature/pressure and in oxygen-free conditions.

Such a result cannot be expected in view of the prior art teachings.

Specifically, it is commonly recognized in certain art (as reported by several scientific papers) that the content of nickel (Ni) in austenitic steels is detrimental under low oxygen condition. Therefore, it is commonly understood that corrosion resistance of duplex stainless steels take advantage from a low content of nickel.

On the contrary, the inventors of the present disclosure have recognized that a certain amount of nickel, lower than in usual austenitic steels but higher than a minimum threshold, has indeed a relatively good impact on corrosion resistance of a duplex stainless steel, if nickel is associated with cobalt (Co) and molybdenum (Mo) according to specific rules.

Specifically, the duplex stainless steels of the disclosure have a content of nickel ranging between 5.5% and 8%, preferably from 6.0% to 7.5% (here and below, all percentages are intended, if not otherwise specified, as weight percentages with respect to the total weight of the steel).

It should be appreciated that nickel is an austenite forming element and a certain amount of nickel is needed to maintain an equilibrium between ferrite and austenite phases. From the other hand, nickel has a negative impact on intermetallic precipitation.

According to the disclosure, cobalt is used in combination with nickel (and replacing part of the nickel) to obtain the required balance between ferrite and austenite phases and to improve corrosion resistance.

In accordance with the present disclosure, the content of nickel can be reduced by replacing nickel with cobalt, that works as a partial substitute and also has the additional advantage of improving the corrosion resistance of the duplex stainless steels having the particular compositions of the disclosure.

It should be appreciated that unlike nickel, cobalt reduces the precipitations of intermetallic phases, strengthens the ferrite matrix and has a positive effect as austenite forming element.

Specifically, the duplex stainless steels of the disclosure have a content of Co in the range between 0.01% and 0.8%, preferably from 0.01% to 0.6%, more preferably from 0.02 to 0.6%, in particular from 0.04% to 0.6%.

According to the disclosure, the contents of nickel and cobalt is also linked to the content of molybdenum.

Molybdenum is a ferrite forming element which accelerates the precipitation of intermetallic phases especially in the presence of relatively high levels of chromium (such as in the duplex stainless steels of the disclosure); therefore, the content of molybdenum should not exceed a maximum threshold.

On the other hand, a certain amount of molybdenum is beneficial for ammonium carbamate corrosion resistance and localized corrosion resistance, especially in the presence of ammonium carbamate and under oxygen-free conditions.

Specifically, molybdenum is in the range between 2% and 2.5%. In certain embodiments, the content of Mo is maintained between 2.0% and 2.4%, in particular between 2.0% and 2.3%.

The features of the disclosure as previously defined also provides a method to configure a duplex stainless steel for use in relatively very corrosive environments, in particular in a urea plant/process.

In particular, the disclosure provides the rules for selecting an effective content of Ni, Co, Mo.

Once selected the content/amount of two out of the three components (Ni, Co, Mo), for example by taking into account the above technical considerations about expected effects of each individual elements, the content/amount of the third component is calculated by applying the relationships of the disclosure.

In addition to Ni, Co and Mo, the duplex stainless steels of the disclosure have a relatively high content of chromium (Cr), which increases corrosion resistance in ammonium carbamate solution environments, and at the same time enables a good microstructure without precipitation of third phases and a good hot workability.

It should be appreciated that chromium has a beneficial effect on corrosion resistance and enables higher process temperatures in urea production applications. Chromium is also beneficial for other types of corrosion such as pitting or crevice. On the other hand, high amounts of chromium increase the possibility of precipitation of intermetallic phases and are detrimental to hot workability. Therefore, the amount of chromium is higher than 30% but lower than 35%, preferably ranging between 30.5 and 35%, more preferably between 30.5 and 33%, even more preferably between 30.5 and 32%, in particular between 30.5 and 31.6%.

The duplex stainless steel of the disclosure may also contain the following elements:

Carbon (C). Carbon generally improves mechanical strength; however, according to the disclosure high contents of carbon are avoided in order to prevent precipitation of carbides. Therefore, the amount of carbon is not higher than 0.03%, preferably from 0.001% to 0.03%, more preferably from 0.001% to 0.02%.

Silicon (Si). Silicon is used as a ferrite forming element and for deoxidization in the steel mill (i.e., in the manufacturing process of the duplex stainless steels). High amounts of silicon are avoided in order to reduce the possibility of precipitation of intermetallic phases. Thus the amount of silicon is not higher than 0.5%, preferably from 0.001% to 0.5%.

Manganese (Mn). Manganese increases the solubility of nitrogen (N), but has also a negative impact on corrosion resistance. Therefore, the amount of manganese is not higher than 2.5%, preferably from 0.001% to 2.5%, more preferably from 0.5% to 2.2%, in particular from 1.0% to 2.2%.

Tungsten (W). Tungsten is a ferrite forming element. Tungsten also enhances general corrosion resistance. In particular, in the same way as Cr, Mo and N, also W increases pitting and crevice resistance. However, W accelerates the precipitation of intermetallic phases so its content is maintained below 2.5%, preferably from 0.001% to 2.5%, more preferably from 0.02% to 1%.

Nitrogen (N). Nitrogen is an austenite forming element. Nitrogen also enhances the microstructure stability delaying the precipitation of intermetallic phases and increases the strength of the metal matrix. Nitrogen is added also to increase the pitting and crevice corrosion resistance. For these reasons, at least 0.3% of nitrogen is used. On the other hand, higher contents of nitrogen would lead to poor hot workability, therefore the maximum value of N content is 0.6%. Thus, the content of N ranges from 0.3 to 0.6%, preferably from 0.35% to 0.6%, in particular from 0.4% to 0.6%.

Copper (Cu). Copper has in general a positive effect depressing the intermetallic precipitation kinetics, especially when relatively high amounts of Mo and W are present. However, for urea production applications copper is a harmful element because it forms complex ions with ammonia and deteriorates corrosion resistance. Therefore, Cu content is limited to a maximum of 1%, preferably from 0.001% to 1%, preferably from 0.001% to 0.9%, more preferably from 0.001% to 0.5%, even more preferably from 0.10 to 0.45% and in particular from 0.10 to 0.40%.

Since the duplex stainless steels of the disclosure have a relatively high content of chromium (as well as nitrogen), hot workability could be negatively affected. In order to facilitate processing (in particular, hot forming) of the duplex stainless steels of the disclosure, one or more of the following elements are optionally added:
Calcium (Ca): 0.004% or less, preferably from 0.001% to 0.004%;
Magnesium (Mg): 0.004% or less, preferably from 0.001% to 0.004%;
One or more rare-earth elements: 0.1% or less, preferably 0.05% or less (total amount).

In various embodiments, the rare-earth elements are selected in the group consisting of Lanthanum (La), Cerium (Ce), Praseodymium (Pr) and mixtures thereof

Rare-earth elements (metals) have relatively very high deoxidation and desulphurization capacities and also decrease the average size of inclusions. They have a beneficial effect on hot workability based on the ability to combine with impurities that can segregate at grain boundaries (such as sulphur) and modify the shape and composition of the inclusions.

The steel compositions of the disclosure may also include unavoidable impurities such as Phosphorus (P) and Sulphur (S). The content of P and S should however be maintained as low as possible. In particular, high amounts of S are detrimental to hot workability. Thus, the S content should be less than 0.005% and the P content should be less than 0.025%. Typical amounts are less than 0.0005% for S and less than 0.020% for P.

The ferrite content of the duplex steel (austeno-ferritic alloy) according to the present disclosure is also of some importance for the corrosion resistance. According to some embodiments, therefore, the ferrite content ranges from 30% to 70% by volume, preferably from 35% by volume to 60% by volume, more preferably from 40% by volume to 60% by volume. The duplex stainless steels of the disclosure are suitably resistant to corrosion even when exposed to ammonium carbamate at high pressure (in particular, at a maximum pressure of 150 bar and higher, preferably of 156 bar and higher, more preferably of 160 bar and higher) and high temperature (in particular 185° C. and higher, preferably 190° C. and higher, more preferably 205° C. and higher), and even in oxygen-free condition.

The disclosure thus provides improved formulations of duplex stainless steels, fully suitable for use in relatively very corrosive conditions such as in a urea environment, (i.e., in contact with a fluid comprising ammonium carbamate), also at temperatures of 185° C. and more (and even at 205° C. and more) and even under oxygen-free conditions.

In particular, the duplex stainless steels of the disclosure are intended for use in contact with ammonium carbamate solutions having a concentration of ammonium carbamate ranging from 15% w to 95% w, in particular from 50% w to 95% w; and/or at a temperature of 185° C. or more, in particular of 190° C. or more, in particular of 205° C. or more).

The highly corrosion resistant duplex stainless steels of the disclosure are suitable for use in any urea environments (i.e., in any kind of urea production plants/processes), and specifically in apparatuses operated at high temperatures (185° C., 190° C. but also 205° C. and higher) in contact with fluids containing ammonium carbamate and also under oxygen-free conditions, such as for instance (but not only) the high pressure strippers used in the ammonia-stripping process or the self-stripping process.

Thus, the duplex stainless steels of the disclosure are especially useful for manufacturing equipment and devices (or parts thereof) which are exposed to concentrated ammonium carbamate at high temperature, such as parts of the heat exchanger tubes and/or, or for example, tubes of strippers.

The duplex stainless steels of the disclosure exhibit relatively excellent corrosion resistance in carbamate solutions (even in oxygen-free condition) also at temperature of 205° C. and higher.

The materials of the disclosure are therefore suitable to be used in a urea production plant of any kinds, including in particular the most demanding conditions of an ammonia-stripping or self-stripping process.

The disclosure thus relates to the use of the duplex stainless steel as disclosed herein in a urea production plant, and specifically in an apparatus, equipment or device (or a part thereof) which is exposed to concentrated ammonium carbamate at high temperature.

The disclosure also relates to an apparatus, equipment or device, in particular of a urea production plant or used in a urea production process, comprising at least a part made of a corrosion resistant duplex stainless steel as disclosed herein.

The disclosure also relates to a plant and a process for the production of urea comprising at least one apparatus, equipment or device having at least a part made of a duplex stainless steel as disclosed herein; and to a method of revamping an existing urea production plant by replacing at least a part of an apparatus, equipment or device of the plant with a part made of a duplex stainless steel as disclosed herein.

As a result of the specific compositions of the duplex stainless steels of the disclosure, the following additional advantages are also achieved over certain of the prior art, in particular in case of use in a high pressure apparatus of the urea plant:
the corrosion rate in an piece of equipment (apparatus/device or part thereof) made of the duplex stainless steels of the disclosure drastically decreases with respect to a piece of equipment made of prior art materials;
the need of passivation air is drastically reduced or even eliminated;
the thickness of the apparatus/device, in particular of the high pressure piping loop, can be reduced, thus resulting in a significant reduction of the total weight and cost of the high pressure section, since the duplex stainless steels of the disclosure also have high mechanical characteristics;
the temperature at the bottom of the stripper can be increased without increasing the corrosion rate; and
it is possible to avoid using, for the high pressure equipment, different materials with different features and prescription in terms of material specifications.

Under particularly severe operation conditions, such as in high pressure strippers used in the ammonia-stripping process or the self-stripping process, corrosion resistance of the duplex stainless steels according to the disclosure can be further increased by coupling the steel with a covering layer made of Zirconium or a Zirconium alloy.

Suitable zirconium materials for such a kind of lining are disclosed, for example, in GB Patent No. 2157687A, EP Patent No. 2310792A1, and EP Patent No. 2427711A2. Therefore, in some embodiments, the duplex stainless steel of the disclosure is provided with a covering layer made of Zirconium or a Zirconium alloy which covers at least a surface portion of the duplex stainless steel.

### BRIEF DESCRIPTION OF THE DRAWINGS

Further features and advantages of the present disclosure will be apparent from the following description of non-limiting embodiments thereof, with reference to the figures of the accompanying drawings, wherein:

FIG. 1 contains a table (Table 1) reporting the composition of exemplary samples of duplex stainless steels according to the disclosure, as well as of some reference samples; and

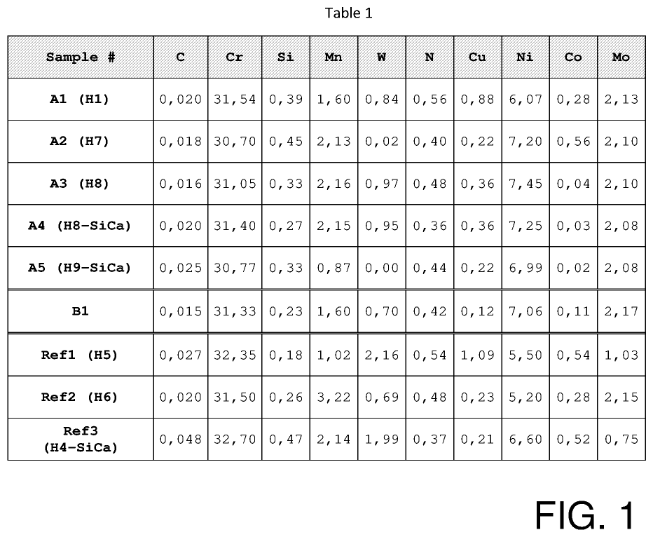

FIG. 2 contains a table (Table 2) reporting the results of corrosion resistance tests performed on the samples of Table 1.

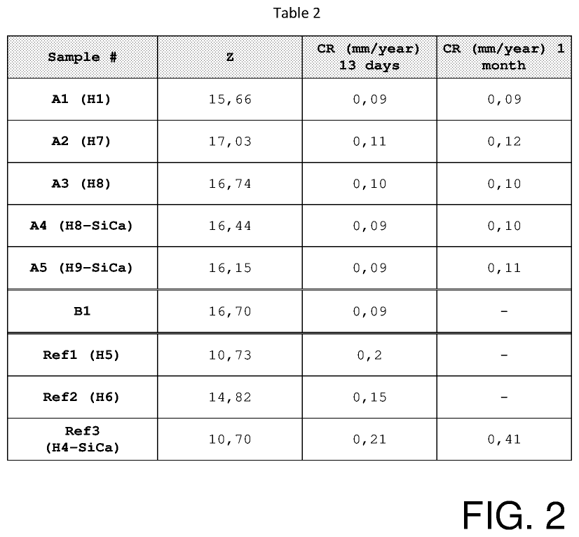

### DETAILED DESCRIPTION

Duplex stainless steels according to the disclosure contain in weight % (% w):
C max 0.03
Si max 0.5
Mn max 2.5
Cr from more than 30.0 to 35.0
Ni 5.5 to 8.0
Co 0.01 to 0.8
Mo 2.0 to 2.5
W max 2.5
N 0.3 to 0.6
Cu max 1.0
and has one or more of:
   Ca max 0.0040
   Mg max 0.0040
   one or more rare-earth elements max 0.1
the balance being Fe and impurities (as commonly understood, impurities are all those elements and compounds which are not purposively added to the steel formulation, but are however present in small amounts being contained in the raw materials used for manufacturing the duplex stainless steel).

The duplex stainless steels of the disclosure are further characterized in that the content of Ni, Co, Mo is such that:

Zₘᵢₙ≤[1.062 (Ni+Co)+4.185 Mo]≤Zₘₐₓ (II)
where:
   Ni, Co, Mo indicate the weight percentage of Ni, Co, Mo respectively;
   Zₘᵢₙ=14.95;
   Zₘₐₓ=19.80.

In other words, the duplex stainless steels of the disclosure have a composition parameter Z, representative of the combined contents of Ni, Co, Mo and defined by formula (I):

Z=1.062 (Ni+Co)+4.185 Mo (I)
where Ni, Co, Mo indicate the weight percentage of Ni, Co, Mo respectively;
and wherein 14.95≤Z≤19.80.

In certain embodiments, the duplex stainless steels according to the disclosure contain in weight % (% w):
C 0.001 to 0.03
Si 0.001 to 0.5
Mn 0.001 to 2.5
Cr from more than 30.0 to 35.0
Ni 5.5 to 8.0
Co 0.01 to 0.8
Mo 2.0 to 2.5
W 0.001 to 2.5
N 0.3 to 0.6
Cu 0.001 to 1.0
and has one or more of:
   Ca max 0.0040
   Mg max 0.0040
   one or more rare-earth elements, in particular selected in the group consisting of La, Ce, Pr and mixture thereof, in a total amount of max 0.1
the balance being Fe and impurities;
and wherein the content of Ni, Co, Mo is such that:

Zₘᵢₙ ≤[1.062 (Ni+Co)+4.185 Mo]≤Zₘₐₓ (II)
where:
Zₘᵢₙ=14.95;
Zₘₐₓ=19.80.

According to the disclosure, the composition parameter Z as above defined ranges between 14.95 and 19.80, preferably between 14.95 and 19.00, more preferably between 14.95 and 18.00, more preferably between 14.95 and 17.50.

### EXAMPLES

Exemplary steel compositions according to the disclosure comprise, in percentages by weight:
C: 0.03% or less;
Si: 0.5% or less;
Mn: 2.5% or less;
Cr: 30.5% to 35%;
Ni: 5.5% to 8%;
Mo: 2% to 2.5%;
W: 0.02% to 1.0%;
Co: 0.01% to 0.8%;
N: 0.3% to 0.6%;
Cu: 1% or less;
one or more of the following:
   Ca: 0.004% or less;
   Mg: 0.004% or less;
   one or more rare earth elements in a total amount of 0.05% or less;
the remainder being Fe and unavoidable impurities;
satisfying the relationship: Z=1.062*(Ni+Co)+4.185*Mo is between 14.95 and 19.80.

Other embodiments of the steel of the disclosure comprise, in percentages by weight:
C: 0.001% to 0.03%;
Si: 0.001% to 0.5%;
Mn: 0.001% to 2.5%;
Cr: more than 30% to 35%;
Ni: 5.5% to 8%;
Mo: 2% to 2.5%;
W: 0.4% to 0.8%;
Co: 0.01% to 0.8%;
N: 0.3% to 0.6%;
Cu: 0.001% to 1%;
one or more of the following:
   Ca: 0.001% to 0.004%;
   Mg: 0.001% to 0.004%;
   one or more rare earth elements in a total amount of 0.001% to 0.1%;
the remainder being Fe and unavoidable impurities;
satisfying the relationship: Z=1.062*(Ni+Co)+4.185*Mo is between 14.95 and 19.80.

Other compositions according to the disclosure comprise, in percentages by weight:
C: 0.001% to 0.03%;
Si: 0.5% or less;
Mn: 0.5% to 2.2%;
Cr: 30.5% to 34%;
Ni: 5.5% to 8%;
Mo: 2% to 2.5%;
W: 2.5% or less;
Co: 0.01% to 0.8%;
N: 0.3% to 0.6%;
Cu: 1% or less;
one or more of the following:
   Ca: 0.004% or less;
   Mg: 0.004% or less;
   one or more rare earth elements in a total amount of 0.05% or less;
the remainder being Fe and unavoidable impurities;
satisfying the relationship: Z=1.062*(Ni+Co)+4.185*Mo is between 14.95 and 19.80.

Yet other compositions according to the disclosure comprises, in percentages by weight:
C: 0.02% or less;
Si: 0.001% to 0.5%;
Mn: 2.5% or less;
Cr: 30.5% to 32%;
Ni: 5.5% to 8%;
Mo: 2% to 2.5%;
W: 0.1% to 1%;
Co: 0.01% to 0.8%;
N: 0.3% to 0.6%;
Cu: 0.15% to 0.25%;
having one or more of the following:
   Ca: 0.004% or less;
   Mg: 0.004% or less;
   La, Ce, Pr or other rare earth elements: 0.05% or less
the remainder being Fe and unavoidable impurities;
satisfying the relationship CRC=1.062*(Ni+Co)+4.185*Mo is between 14.95 to 19.80.

Other compositions according to the disclosure comprises, in percentages by weight:
C: 0.03% or less;
Si: 0.5% or less;
Mn: 0.001% to 2.2%;
Cr: 31% to 35%;
Ni: 6% to 7.5%;
Mo: 2% to 2.5%;
W: 2.5% or less;
Co: 0.01% to 0.8%;
N: 0.4% to 0.6%;
Cu: 0.9% or less;
one or more of the following:
   Ca: 0.004% or less;
   Mg: 0.004% or less;
   one or more rare earth elements in a total amount of 0.05% or less;
the remainder being Fe and unavoidable impurities;
satisfying the relationship: Z=1.062*(Ni+Co)+4.185*Mo is between 14.95 and 19.80.

Other examples according to this disclosure comprise, in percentages by weight:
C: 0.03% or less;
Si: 0.5% or less;
Mn: 0.5% to 2.2%;
Cr: 30.5% to 35%;
Ni: 5.5% to 6.5%;
Mo: 2% to 2.5%;
W: 0.001% to 2.5%;
Co: 0.01% to 0.6%;
N: 0.35% to 0.6%;
Cu: 1% or less;
one or more of the following:
   Ca: 0.004% or less;
   Mg: 0.004% or less;
   one rare earth element selected from La, Ce, Pr or a combination thereof: 0.05% or less
the remainder being Fe and unavoidable impurities;
satisfying the relationship: Z=1.062*(Ni+Co)+4.185*Mo is between 14.95 and 19.80.

For example, the present disclosure relates to elementary steel compositions that comprise, in percentages by weight:
C: 0.03% or less;
Si: 0.5% or less;
Mn: 2.2% or less;
Cr: 31% to 32%;
Ni: 5.5% to 8%;
Mo: 2% to 2.5%;
W: 2.5% or less;
Co: 0.02% to 0.4%;
N: 0.3% to 0.6%;
Cu: 0.001% to 1%;
one or more of the following:
   Ca: 0.004% or less;
   Mg: 0.004% or less;
   one rare earth element selected from La, Ce, Pr or a combination thereof: 0.05% or less
the remainder being Fe and unavoidable impurities;
satisfying the relationship: Z=1.062*(Ni+Co)+4.185*Mo is between 14.95 and 19.80.

Other exemplifying composition according to the disclosure comprise, in percentages by weight:
C: 0.03% or less;
Si: 0.5% or less;
Mn: 2% or less;
Cr: 30.5% to 33%;
Ni: 5.5% to 8%;
Mo: 2% to 2.5%;
W: 0.2% to 1%;
Co: 0.02% to 0.4%;
N: 0.3% to 0.6%;
Cu: 1% or less;
one or more of the following:
   Ca: 0.001% to 0.004%;
   Mg: 0.001% to 0.004%;
   La, Ce, Pr or other rare earth elements: 0.001% to 0.05%
the remainder being Fe and unavoidable impurities;
satisfying the relationship: Z=1.062*(Ni+Co)+4.185*Mo is between 14.95 and 19.80.

Further example compositions according to the disclosure comprise, in percentages by weight:
C: 0.02% or less;
Si: 0.5% or less;
Mn: 0.5% to 2.2%;
Cr: 30.5% to 34%;
Ni: 5.5 to 8%;
Mo: 2 to 2.5%;
W: 0.02 to 1%;
Co: 0.02 to 0.6%;
N: 0.3 to 0.6%;
Cu: 0.20% to 0.9%;
one or more of the following:
   Ca: 0.004% or less;
   Mg: 0.004% or less;
   one or more rare earth elements in a total amount of 0.05% or less;
the remainder being Fe and unavoidable impurities;
satisfying the relationship: Z=1.062*(Ni+Co)+4.185*Mo is between 14.95 and 19.80.

In particular, duplex stainless steels having the compositions in Table 1 were prepared and tested (in Table 1, some components are not indicated, being however in the amounts as previously disclosed).

The samples were prepared as common in the field and tested according to standard testing procedure. Samples A1 to A5 were prepared by using laboratory produced materials, while sample B1 was prepared by using material from an industrial production.

In particular, corrosion tests were performed in a high pressure autoclave in ammonium carbamate solution at high pressure and high temperature (conditions representative of typical operation conditions in urea plants, in particular in the tubes of a urea stripper).

In particular, the corrosion resistance of the duplex stainless steels of the disclosure was tested in an oxygen-free carbamate solution, having a composition simulating the worst conditions normally occurring in the tubes of a high pressure section urea stripper of a urea plant, and at a temperature of 208° C.

In more detail: the corrosion behavior of the laboratory heats was checked via immersion tests that were conducted in a 5-liter Zirconium autoclave. The autoclave was equipped with adequate feed and discharge lines and a stirrer. The test solution contained a mixture of urea, ammonia and water, at concentrations similar to those of the urea synthesis process. Temperature and pressure for the experiments were set in the upper level of the typical ranges measured in a urea stripper, 180-210° C. and 140-200 bar, respectively. The test solution was degassed before starting the tests to eliminate oxygen from the system. These experiments were designed to simulate the most severe conditions in a stripper of a urea plant without oxygen injection; note that under current working conditions in a urea plant, the stainless steel would perform even better, due to the presence of low amounts of oxygen and less aggressive conditions.

Test duration was 13 and 30 days. ASTM G31 (Standard Practice for Laboratory Immersion Corrosion Testing of Metals) standard indications were followed for test specimen preparation and the corrosion rate was measured by the gravimetric method.

After exposures of 13 days and 30 days respectively in the oxygen-free carbamate solution, the corrosion resistance was evaluated by calculating the corrosion rate (expressed in mm/year).

The results are shown in Table 2.

The results confirm that the samples (A1-A5; B1) made of a duplex stainless steel according to the disclosure (i.e., satisfying the composition requirements of the disclosure (in particular with respect to the combined content of Ni, Co, Mo), have a corrosion rate significantly lower than comparative samples Ref1, Ref2, Ref3 and thus a better corrosion resistance).

It should be appreciated that the experimental tests confirm that when Z satisfies the requirement: 14.95≤Z≤19.80, corrosion values are significantly lower than those exhibited by reference materials.

Corrosion values would be even significantly lower in working conditions in a urea plant, since the experimental set-up conditions are much more aggressive.

Finally, although the disclosure has been disclosed in relation to the above-mentioned embodiments, it is to be understood that many other possible modifications and variations can be made without departing from the scope of the appended claims. Accordingly, various changes and modifications to the presently disclosed embodiments will be apparent to those skilled in the art.

1-31. (canceled)

32. A urea production process comprising:
at least one step performed in a device comprising duplex stainless steel containing:
carbon in weight percentage of 0.03% or less,
silicon in weight percentage of 0.5% or less,
manganese in weight percentage of 2.5% or less,
chromium in weight percentage from more than 30.0% to 35.0%,
nickel in weight percentage from 5.5% to 8.0%,
cobalt in weight percentage from 0.01% to 0.8%,
molybdenum in weight percentage from 2.0% to 2.5%,
tungsten in weight percentage of 2.5% or less,
nitrogen in weight percentage from 0.3% to 0.6%,
copper in weight percentage of 1.0% or less,
at least one of:
calcium in weight percentage of 0.0040% or less,
magnesium in weight percentage of 0.0040% or less, and
at least one rare-earth element in a total amount in weight percentage of 0.1% or less, and
a balance being iron and any impurities;
wherein the duplex stainless steel has a composition parameter representative of the combined contents of nickel, cobalt, molybdenum defined by a formula of:
composition parameter=1.062 (nickel+cobalt)+4.185 molybdenum
wherein in the formula, nickel indicates the weight percentage of nickel, cobalt indicates the weight percentage of cobalt, molybdenum indicates the weight percentage of molybdenum, and the composition parameter ranges from between 14.95 and 19.80.

33. The urea production process of claim 32, wherein the duplex stainless steel contains from 0.001% to 0.02% in weight percentage of carbon.

34. The urea production process of claim 32, wherein the duplex stainless steel contains from 30.5% to 35% in weight percentage of chromium.

35. The urea production process of claim 32, wherein the duplex stainless steel contains from 30.5% to 32% in weight percentage of chromium.

36. The urea production process of claim 32, wherein the duplex stainless steel contains from 30.5% to 31.6% in weight percentage of chromium.

37. The urea production process of claim 32, wherein the duplex stainless steel contains from 0.001% to 0.9% in weight percentage of copper.

38. The urea production process of claim 32, wherein the duplex stainless steel contains one of:
from 0.10% to 0.90% in weight percentage of copper, and
from 0.10% to 0.40% in weight percentage of copper.

39. The urea production process of claim 32, wherein the duplex stainless steel contains from 0.02% to 0.6% in weight percentage of cobalt.

40. The urea production process of claim 32, wherein the duplex stainless steel contains from 6.0% to 7.5% in weight percentage of nickel.

41. The urea production process of claim 32, wherein the duplex stainless steel contains from 0.5% to 2.5% in weight percentage of manganese.

42. The urea production process of claim 32, wherein the duplex stainless steel contains from 0.5% to 2.2% in weight percentage of manganese.

43. The urea production process of claim 32, wherein the duplex stainless steel contains from 0.001% to 2.5% in weight percentage of tungsten.

44. The urea production process of claim 32, wherein the duplex stainless steel contains from 0.02% to 1.0% in weight percentageof tungsten.

45. The urea production process of claim 32, wherein the duplex stainless steel contains from 0.001% to 0.004% in weight percentage of calcium.

46. The urea production process of claim 32, wherein the duplex stainless steel contains from 0.001% to 0.004% in weight percentage of magnesium.

47. The urea production process of claim 32, wherein the duplex stainless steel contains at least one rare-earth element in a total amount of 0.05% or less.

48. The urea production process of claim 32, wherein the duplex stainless steel contains at least one rare-earth element selected from the group consisting of: lanthanum, cerium, praseodymium and at least one mixture thereof.

49. The urea production process of claim 32, wherein the duplex stainless steel contains, as impurities, no more than at least one of: 0.025% in weight percentage of of phosphorus (P) and 0.005% in weight percentage of sulphur.

50. The urea production process of claim 32, wherein the duplex stainless steel is in contact with an ammonium carbamate solution having a concentration of ammonium carbamate ranging from 15% in weight percentage to 95% in weight percentage.

51. The urea production process of claim 32, wherein the duplex stainless steel is in contact with an ammonium carbamate solution having a concentration of ammonium carbamate ranging from 50% in weight percentage to 95% in weight percentage.

52. The urea production process of claim 32, wherein the duplex stainless steel is in contact with an ammonium carbamate solution at a temperature of at least 185° C.

53. The urea production process of claim 32, wherein the duplex stainless steel is in contact with an ammonium carbamate solution at a temperature of at least 205° C.

54. The urea production process of claim 32, wherein the duplex stainless steel is used in at least one of: at a maximum temperature of at least 185° C., at a maximum pressure of at least 150 bar, and in an environment having an ammonia/carbon dioxide molar ratio in the range of 3.2 to 3.6.

55. The urea production process of claim 32, wherein the duplex stainless steel is used in at least one of: at a maximum temperature in a range of 205° C. to 215° C., at a maximum pressure of at least 160 bar, and in an environment having a ammonia/carbon dioxide molar ratio in the range of 3.2 to 3.6.

56. The urea production process of claim 32, wherein the at least one step performed in the device comprising duplex stainless steel occurs in a urea environment under oxygen-free conditions.

57. The urea production process of claim 32, wherein the at least one step performed in the device comprising duplex stainless steel comprises using the duplex stainless steel in a high pressure section of a urea plant.

58. The urea production process of claim 32, wherein the at least one step performed in the device comprising duplex stainless steel comprises using the duplex stainless steel in association with performing one of: an ammonia-stripping process and a self-stripping process.

59. The urea production process of claim 32, wherein the at least one step performed in the device comprising duplex stainless steel comprises using the duplex stainless steel in a high pressure stripper configured for one of: ammonia-stripping and self-stripping.

60. The urea production process of claim 32, wherein the duplex stainless steel is coupled with a covering layer which covers at least a surface portion of the duplex stainless steel, the covering layer being made of one of: zirconium and a zirconium alloy.

61. The urea production process of claim 32, wherein the composition parameter ranges between 14.95 and 17.50.

62. A urea production plant device comprising:
a duplex stainless steel containing:
carbon in weight percentage of 0.03% or less,
silicon in weight percentage of 0.5% or less,
manganese in weight percentage of 2.5% or less,
chromium in weight percentage from more than 30.0% to 35.0%,
nickel in weight percentage from 5.5% to 8.0%,
cobalt in weight percentage from 0.01% to 0.8%,
molybdenum in weight percentage from 2.0% to 2.5%,
tungsten in weight percentage of 2.5% or less,
nitrogen in weight percentage from 0.3% to 0.6%,
copper in weight percentage of 1.0% or less,
at least one of:
calcium in weight percentage of 0.0040% or less,
magnesium in weight percentage of 0.0040% or less, and
at least one rare-earth element in a total amount in weight percentage of 0.1% or less, and
a balance being iron and any impurities;
wherein the duplex stainless steel has a composition parameter representative of the combined contents of nickel, cobalt, molybdenum defined by a formula of:
composition parameter=1.062 (nickel+cobalt)+4.185 molybdenum
wherein in the formula, nickel indicates the weight percentage of nickel, cobalt indicates the weight percentage of cobalt, molybdenum indicates the weight percentage of molybdenum, and the composition parameter ranges between 14.95 and 19.80.

63. A method of modifying a urea production plant, the method comprising:
replacing at least part of the urea production plant with a device comprising a duplex stainless steel containing:
carbon in weight percentage of 0.03% or less,
silicon in weight percentage of 0.5% or less,
manganese in weight percentage of 2.5% or less,
chromium in weight percentage from more than 30.0% to 35.0%,
nickel in weight percentage from 5.5% to 8.0%,
cobalt in weight percentage from 0.01% to 0.8%,
molybdenum in weight percentage from 2.0% to 2.5%,
tungsten in weight percentage of 2.5% or less,
nitrogen in weight percentage from 0.3% to 0.6%,
copper in weight percentage of 1.0% or less,
at least one of:
calcium in weight percentage of 0.0040% or less,
magnesium in weight percentage of 0.0040% or less, and
at least one rare-earth element in a total amount in weight percentage of 0.1% or less, and
a balance being iron and any impurities;
wherein the duplex stainless steel has a composition parameter representative of the combined contents of nickel, cobalt, molybdenum defined by a formula of:
composition parameter=1.062 (nickel+cobalt)+4.185 molybdenum
wherein in the formula, nickel indicates the weight percentage of nickel, cobalt indicates the weight percentage of cobalt, molybdenum indicates the weight percentage of molybdenum, and the composition parameter ranges between 14.95 and 19.80.
